(19) United States
(12) Patent Application Publication
(10) Pub. No.: US 2004/0054344 A1
(43) Pub. Date: Mar. 18, 2004

(54) Absorbent article comprising a fluid distribution layer and a targeted fluid transfer induced by pressure

(76) Inventors: Henning Roettger, Braunschweig (DE); Joachim Bauer, Eggenwil (CH); Indra Roy, Eggenwil (CH)

Correspondence Address: WILLIAM COLLARD, COLLARD & ROE, P.C., 1077 NORTHERN BOULEVARD, ROSLYN, NY, 11576, US

(21) Appl. No.: 10/415,598

(22) PCT Filed: Oct. 31, 2001
(86) PCT No.: PCT/EP01/12626

(30) Foreign Application Priority Data
Oct. 31, 2000 (DE) 100 53 895.9

(51) Int. Cl. A61F 13/15; A61F 13/20

(52) U.S. Cl. 604/378

(57) ABSTRACT

The invention relates to a disposable product (1), in particular a hygiene product which absorbs fluids. Said product comprises an upper layer (2) and a core (4) which absorbs and stores fluids. A fluid distribution and storage layer (6) is located below the core (4), in order to absorb fluid that escapes from the absorbent core as a result of pressure being exerted on said core (4).

The present invention relates to a fluid-acquiring disposable product, for example a single-use product, especially a hygiene product. The product has a topsheet ply and a fluid-acquiring and -storing core.

It is a well-known problem to retain fluid in a hygiene product. EP 1 022 009 A2 discloses a three-ply backsheet ply for a hygiene product where a middle ply of high density is disposed between first and second plies. The three-ply backsheet ply is said to prevent fluid leaking from the hygiene product. U.S. Pat. No. 5,928,209 reveals a backsheet ply which is constructed in multiply fashion from various nonwoven layers. The backsheet ply forms a barrier layer for liquid, but is said to be water vapor pervious at the same time. U.S. Pat. No. 5,643,239 discloses a diaper construction comprising a topsheet ply, a core, a barrier ply and a backsheet ply. The barrier ply and the backsheet ply are water vapor pervious and formed such that they form a barrier with regard to liquid. A barrier action with regard to liquid is said to be enhanced by this prior art by disposing a further hydrophobic enhancer ply formed of nonwoven between the barrier ply and the hydrophobic backsheet ply.

It is an object of the present invention to provide a fluid-acquiring breathable disposable product, for example a single-trip article, especially a hygiene product, which is capable of substantially preventing passage of fluid therethrough under service conditions.

This object is achieved by a fluid-acquiring disposable product having the features of claim 1 and also by a ply for a fluid-acquiring disposable product having the features of claim 13.

Further advantageous embodiments and refinements are indicated in dependent claims.

A fluid-acquiring disposable product and especially hygiene product has a topsheet ply and a fluid-acquiring and -storing core. Outwardly of the core there is disposed a fluid distribution and storage ply to acquire fluid which exits from the fluid-acquiring core under pressure exerted thereon.

The fluid distribution and storage ply is capable of storing the fluid exiting from the core. When pressure is exerted on only one part of the core and hence also on only one part of the outwardly disposed fluid distribution and storage ply, the fluid preferentially spreads out sideways in the fluid distribution and storage ply instead of then exiting from the outwardly disposed ply as well. The fluid distribution and storage ply will hereinbelow be referred to as fluid distribution ply.

In one embodiment, the fluid distribution ply exhibits a tower flow resistance under pressure on an area of its core with regard to any distribution of the fluid in the fluid distribution ply compared with that to fluid exiting from the fluid distribution ply. This embodiment eliminates the risk of soiled underwear for example when pressure is exerted on the fluid-storing core when seated for example. The fluid distribution ply makes it possible that pressure peaks acting on the core and streams of fluid exiting from the core as a result thereof be absorbed and stored. Preferably, the fluid is only interveningly stored and flows back into the core on pressure being removed. In a further refinement, the fluid distribution ply has a volume capable of ensuring intervening storage in this volume alone. Individual fibers of the fluid distribution ply are preferably not capable of acquiring fluid. More particularly, the fibers are hydrophobic.

In a further refinement, a ply adjoining the fluid distribution ply has a higher hydrostatic pressure resistance than the fluid distribution ply. For example, the fluid flow resistance can be enhanced by one or more barrier plies. The barrier ply can be disposed between the core and the fluid distribution ply, outwardly of the fluid distribution ply or else both. The adjoining ply can be a barrier ply as used in the above-described prior art or else a water vapor pervious film. Preferably, the fluid distribution ply has a larger average pore diameter than a ply adjoining the fluid distribution ply. First, the fluid distribution ply can have a larger holding volume as a result. Secondly, controlling pore number and size makes it possible to achieve a specifically targeted pressure reduction in the fluid-acquiring disposable product exposed to applied pressure and flowing fluid.

In a further embodiment, the construction of the fluid distribution ply is such that the fluid emitted by the core is distributed within the fluid distribution ply. The pressure exerted on the core is simultaneously also exerted on the fluid distribution and storage ply. When the core is compressed and fluid escapes, the fluid distribution ply offers the fluid a lower resistance to distribution in this ply itself in the exit region. In a further refinement, the fluid distribution ply is formed to avoid fluid getting as far as any backsheet ply possibly outwardly disposed with regard to the fluid ply. In a further embodiment, the fluid does get as far as the backsheet ply. There, however, the fluid is distributed sideways in the fluid distribution ply instead of passing through the backsheet ply. The backsheet ply forms an outer ply of the disposable product.

The fluid distribution ply has the further advantage that it provides at least vapor perviousness coupled with simultaneous protection against wetting through. Preferably, the fluid distribution ply is air pervious also. Similarly, an optional outwardly disposed backsheet ply can be water vapor pervious and/or air pervious. A porous film can be used, for example.

In a further embodiment, the fluid distribution ply has a nonunitary construction. This nonunitary construction is achievable for example by the fluid distribution ply having a first average pore size and a second average pore size, the first pore size differing from the second pore size. It is similarly possible for the fluid distribution ply to have regions having different densities and/or differently sized average interspaces.

These regions may seamlessly merge into one another or else be separated from each other via interfaces for example. The fluid distribution ply may further comprise regions having different fiber and/or filament diameters. In a further refinement, the fluid distribution ply comprises two or more layers which are firmly bonded together for example. Furthermore, the wettability of the fluid distribution ply may be controlled, for example via the material used, via an appropriate coating or wetting or other. The wettability may also differ across a cross section through the fluid distribution ply, for example be reduced in the direction of an outer surface of the disposable product. In an embodiment of the fluid distribution ply, the fluid distribution ply comprises a gradient with regard to the available volume across a cross section through the fluid distribution ply. Materials for the fluid distribution ply may be fibers, especially nonwovens, but also foams and also mixtures thereof.

Preferably, the fluid distribution ply differs in various regions to such an extent that an applied pressure is nonuniformly distributed in the fluid distribution ply. This permits specifically targeted distribution of the fluid: when the resistance in the direction of the backsheet ply increases, the fluid escapes sideways. The fluid distribution ply comprises a sufficient storage volume for this. In a further refinement, the fluid distribution ply has a higher flow resistance in use in a first territory of preferred pressure application of the fluid-acquiring disposable product compared with a second, adjacent territory of the fluid distribution ply.

In a further refinement, the fluid distribution ply comprises a certain stiffness. To this end, the material used for the fluid distribution ply may comprise a main direction of stiffness. This main direction of stiffness preferably extends substantially along the thickness of the fluid distribution ply. In a further embodiment, the core has a lower stiffness than the fluid distribution ply. Preferably, the fluid distribution ply has a highly porous structure which allows fluid present in the fluid distribution ply more freedom to move compared with fluid present in the core. The core for example additionally comprises superadsorbent, cellulose material or other. In a further refinement, the core exhibits capillary forces on a liquid, whereas the fluid distribution ply preferably possesses only extremely small if any capillary forces.

The disposable product, for example a diaper, a sanitary napkin or the like, is ergonomically adapted to its use. As it is being worn, the disposable product experiences various states. One of these states is the exertion of increased pressure, for example when the wearer is seated or leans the affected body part against a surface in conjunction with the disposable product. It is foreseeable on the basis of ergonomic considerations that in the case of diapers for example certain regions of the buttocks will be particularly exposed to pressure. Appropriately adapting resistances of the fluid distribution ply to this sitting characteristic of the disposable product provides a corresponding one of fluid flowing out of the core into the fluid distribution ply.

In a further embodiment, the fluid distribution ply comprises a capillary effect. This capillary effect preferably develops in the fluid distribution ply at a distance from the core. For example, the capillary effect is at least partly directed in the direction of the backsheet ply. From there, in a further refinement, the capillary effect may act such that the fluid is distributed sideways. The fluid distribution ply is preferably engineered in such a way that the capillary effect is such that the fluid distribution ply surface which directly or indirectly adjoins the core initially only acquires exiting fluid. Appropriate channeling and distribution within the fluid distribution ply ensures that the capillary effect comes into action only at some distance from the core. The capillary effect may be achieved through appropriate shaping of the fibers or filaments or other materials of the fluid distribution ply. Preferably, the fluid distribution ply has a capillary action in a lateral region whereby the fluid is led back to the core from the direction of a potentially existing backsheet ply. The core itself likewise has a capillary action, in a further refinement. This capillary action is preferably effective in the immediate vicinity of the fluid distribution ply. Fluid may thus be interveningly stored in the fluid distribution ply and subsequently, given appropriate distribution, flow back into the core.

In a further refinement, the fluid distribution ply has a mode of action on impinging fluid that, based on a cross section, may be described in terms of a first region which has a hydrophobic action, a second region which has a hydrophilic action and a third region which has a hydrophobic action.

To prevent sideways exit of fluid from the core, the fluid distribution ply advantageously projects beyond the core to the side. To this end, the fluid distribution ply may encase the core at least partly or else wholly.

By a further concept, which may also be pursued independently with regard to another nonwoven fabric, for example a composite formed from spunbond, meltblown and/or spunbond (SMS material), the fluid distribution ply comprises a flow inhibitor. The inhibitor is preferably activated on contact with a fluid. The flow inhibitor may be activated for example by agglomeration bodies to which the fluid becomes bound or through which a solidification is initiated within the fluid. Such a process is also achievable through an appropriate nucleating action which can be introduced into the fluid on flowing into and flowing through the fluid distribution ply. Various substances may be used depending on the disposable product's field of use. When the disposable product is used in the form of a hygiene product which comes into contact with a body fluid, especially blood, it is possible to use substances which act on proteins, hemoglobin or other constituents and are activatable in particular. These substances may cause the blood to coagulate for example.

It is further possible for the flow inhibitor to have a wicking effect. The fluid becomes bound to the inhibitor by virtue of this wicking function. The inhibitor itself may be swellable, as is the case with superabsorbent materials for example. It is likewise possible to use odor-controlling material or other materials which have a specific action, for example zeolites, silicates, pH regulators and/or carbon.

Preferably, the fluid distribution ply is equipped with a fluid-acquiring volume which is for example between 1/10 and ⅓ of that of the core. A larger volume may be provided as well as a smaller volume. This is dependent for example on the use of the disposable product, of the construction and the different properties of the individual plies used. Preferably, the actual fluid acquisition and fluid storage occur in the core. It is only in special circumstances, such as for example pressure on the core and fluid exiting as a result, that the fluid distribution ply assumes its function as a store. By providing an appropriate volume, it may further be ensured at the same time that the fluid distribution ply and its storage function shall also provide an emergency reserve volume. This can be necessary for example when the core is completely saturated. Excess fluid is appropriately stored by the fluid distribution ply without fluid being transmitted to the possibly provided backsheet ply. Such a state occurs for example in the event of a prolonged wear time of the disposable product as a hygiene product, in the case of a sanitary napkin, or in the case of a supplied volume of fluid that is above certain limits. This is in connection with a stream of fluid arriving within a short period. The fluid distribution ply is preferably used in diapers for neonates, ie for the first few weeks after birth. The construction of the individual plies is preferably such that some or all of the fluid in the fluid distribution ply flows back into the core after pressure has been removed. This may be accomplished for example by exploiting the tendency of the fluid to establish an equilibrium distribution.

The function of the fluid distribution ply is supported by disposing between it and the core a barrier ply which hereinbelow will be referred to as barrier layer. This barrier layer forms a resistor between the core and the fluid distribution ply. In one embodiment, the fluid succeeds in passing through the barrier layer into the fluid distribution ply only on exceedence of an externally applied pressure. The barrier layer preferably has a flow resistance to fluid that is greater than the flow resistance to this fluid possessed by the fluid distribution ply itself. The fluid-pervious barrier layer is for example a nonwoven fabric such as for example an SMS material, an SM ply, a carded material, a spunbond ply or other prior art barrier materials such as for example a film, a microporous film or a composite formed from film and nonwoven fabric or the like.

In a further embodiment, which may also be combined with others, the disposable product comprises a barrier layer comprising a water-impervious film adjoined by a fluid distribution ply. The pressured fluid is unable to pass through the film and exits sideways from the core. At least in this region there is disposed at least one fluid distribution ply, for example in the form of a strip or as a partial or complete sheath around at least one lateral end of the core. The fluid distribution ply or plies preferably store the fluid interveningly. After pressure has been removed from the core, the fluid can flow back out of the fluid distribution ply into the core.

In a further refinement, a backsheet ply of the disposable product and/or a barrier layer is at least partly hydrophobic.

By a further concept, the invention also provides a ply for a fluid-acquiring disposable product. The ply is a fluid distribution and storage ply as described above. Preferably, this ply has a flow resistance which is larger in one thickness section of the ply than in a longitudinal ply section directly adjacent to the thickness section in order that fluid may be distributed in the fluid distribution ply. Such a flow resistance eliminates the preferential tendency of the fluid entering the ply to pass through this ply. The flow resistance is achieved for example through a nonuniform construction for the fluid distribution and storage ply. Means to this end are exemplified above.

A further way to achieve controlled fluid management is for the ply to comprise a bonding pattern which influences and preferably controls the flow resistance. The bonding pattern may define a region without bonding sites. This region preferably has a lower flow resistance and preferably also a lower distribution resistance. For example, a region adjoining a bonding region having an appropriate bonding pattern has narrower pores or narrower interspaces because of the bonding sites. These pores or interspaces increase the flow resistance, since the bonding sites themselves reduce the fraction of pervious area. The bonding pattern itself may be pointwise, linear, herringbonelike, large in area, small in area, mixtures thereof or the like. It may be formed by means of appropriately patterned calendering rolls, by means of ultrasound processes, by means of adhesives, by means of the material of the fibers or filaments themselves or other tools or materials. The bonding may also be controlled such that a capillary effect is thereby achieved. Preferably, a bonding pattern is used to adjust the flow resistance in the direction of the sides of the ply.

In one embodiment of the ply as a fluid distribution and storage ply for a hygiene product, the ply is at least 0.05 mm, especially at least 0.1 mm and preferably 0.5 mm in thickness. It may also be more than 1 mm in thickness. Thickness may also vary as a function of location.

The invention further provides a process for testing a flow distribution and storage ply for a fluid-acquiring disposable product as described above or for a ply described above. The process comprises the steps of:

applying a fluid to a multi-ply structure which comprises the fluid distribution ply, the fluid distribution ply adjoining a test surface which is fluid acquiring,

applying a defined pressure of at least 2000 N/m² to the multi-ply structure for a period of at least 5 minutes, and

determining at least the fluid passage into the fluid distribution ply.

In a further step, not just the fluid passage is measured and optionally assessed. On the contrary, the degree of soiling is also assessed on the basis of the fluid passage through the fluid ply. This is preferably accomplished as a result of the multi-ply construction adjoining the test surface. The defined test conditions make it possible to ascertain whether the test surface comprises any soiling. This soiling is due to fluid exiting from the core and/or fluid exiting from the fluid distribution ply. This makes it possible to verify whether a hygiene product in contact with underwear is not going to soil the latter.

The test can be varied by varying the time as well as the pressure. For example, the pressure may about 22 kN/m² and the time about 10 minutes. Preferably, the pressure increases as the pressure period shortens.

Furthermore, the fluid distribution and storage ply may comprise the following properties singly or combined, especially moreover in combination with properties of the core:

odor control, fluid adhesion, fluid dehesion, alcohol repulsion, hydrophilicity, hydrophobicity, dilution of fluid, thickening of fluid, solidification of fluid, coagulation of fluid, precipitation or drying of fluid.

More particularly, the fluid distribution ply may comprise movement incapacitators and preferably immobilizers, for example as described above, at certain regions for example in the edge region. This eliminates fluid exiting from the fluid distribution ply. It is also possible to provide an appropriate property in the fluid distribution ply in an edge region which is directed toward the backsheet ply. This edge region has the function to act as a barrier, for example by closing openings which are otherwise pervious to water vapor at least. It is further possible for such a movement incapacitator or immobilizer to be disposed in the fluid distribution ply such that the fluid rendered movement incapable as a result of the movement incapacitator or immobilizer produces a specific increase in flow resistance. For example, a fluid-saturated region can be sealed off thereby. Additional fluid flow is thereupon rerouted or redirected into other regions of the fluid distribution ply. For example, the fluid distribution ply may have been partly or wholly given an appropriate treatment, for example by means of oleophobic treatment, a hydrophobic treatment and/or a surface tension lowering treatment, for example an HO+ treatment. The effects of the treatment can be physically/chemically intramolecular in particular.

Preferably, the material which may also be referred to as function-conferring substance is incorporated into and/or applied atop the fluid distribution ply in accordance with a predeterminable pattern at mutually spaced apart points or locations. These points or sites are selected such that for example a property such as air perviousness of the fluid distribution ply is only minimally disrupted, if at all, in the event of the substance being activated. For example, the fluid distribution ply comprises sufficient additional substance for air perviousness to decrease by at most 70% and especially by 50% when the fluid distribution ply is saturated compared with a fluid distribution ply without stored fluid. Air perviousness is preferably measured by the air permeability test method ERT 140.1-81. The air perviousness of the fluid distribution ply is preferably greater than that of the backsheet ply. The air perviousness of the backsheet ply is up to 3000 l/m²s in one illustrative embodiment. When an additional barrier layer is disposed intermediate the core and the fluid distribution ply, the barrier layer preferably has an air perviousness of up to 1000 l/m²s. In a further illustrative embodiment, the backsheet ply has a DIN 53886 hydrohead value of at least 8 cm, while the barrier layer has a value of at least 7 cm. The hydrohead value of the fluid distribution ply is preferably lower than the lowest of the two values, while the hydrohead value of the barrier layer is preferably greater than that of the backsheet ply.

An abovementioned average pore size distribution and the available storage volume of the fluid distribution and storage ply are preferably determined via a Coulter Porometer 2. In one embodiment, the average pore size and also the total storage volume are preferably greater than in the case of the backsheet ply and also in the case of the barrier layer.

In a further refinement, the fluid distribution and storage ply has a pressure resistance which is different to those of the barrier layer and/or the backsheet ply. The pressure resistance indicates the behavior of the respective ply in response to mechanically applied pressure from the outside. The pressure resistance is measured using a Schiefer compressometer for example. In one embodiment, the pressure resistance of the fluid distribution ply is less than that of the barrier layer and of the backsheet ply. In another embodiment, the pressure resistance of the fluid distribution ply is greater than at least that of the barrier layer or at least of the backsheet ply. More particularly, the fluid distribution ply may also have a different pressure resistance than the core. Preferably, the pressure resistance of the core is less than that of the fluid ply.

If, for example, a fluid distribution and storage ply is disposed intermediate a barrier ply and a backsheet ply which both serve as fluid barriers, the intermediately disposed fluid distribution ply will on average have larger pores than at least one of the two other plies. Preferably, the pores are even larger than those of both plies. On pressure being exerted on one of the two plies, the fluid will migrate into the fluid distribution ply instead of being forced through the backsheet ply. Preferably, the fluid distribution ply is partly or wholly hydrophilic on the inside or on the surface in order that distribution may be augmented.

In a further refinement, the barrier ply which separates the core from the fluid distribution ply is at least substantially liquid impervious, for example by means of a microporous film which is pervious to water vapor, by means of an air-pervious film or by means of an aporous film. In another refinement, the barrier ply is rendered liquid pervious, for example by use of a hydrophilic nonwoven, a spunbond, a carded or else an SMS nonwoven fabric. It is likewise possible to use an air-pervious film which has been apertured. The aperturing has preferably been carried out such that conelike structures are formed that have an opening at the tip. The opening can be closed on pressure being exerted on the tip. The cone is preferably disposed in the direction of the core.

When partial pressure is exerted, fluid which has penetrated into the fluid distribution ply will escape sideways into less highly pressure-stressed regions. Preferably, any resistance in the fluid distribution ply is not higher than the resistance offered to the fluid as it passes through the largest pore of a barrier ply, especially of the backsheet ply. In a further refinement, the fluid distribution ply remains air pervious after acquisition of fluid and/or under pressure. In another embodiment, the fluid distribution ply comprises a film which is microporous and/or apertured. The film preferably has, at least on one surface, small hairs which may be solid or in particular hollow. The hairs are for example at least 250 μm and preferably about 450 μm in length. In a first embodiment, the hairs are disposed on a film surface not facing the core. In a second embodiment, hairs are disposed on both surfaces of the film.

In the case of a sanitary napkin product for example, the fluid distribution ply preferably has a wet-through value (as termed by Corovin's wet-through test method) which is not more than 0.5 g and more preferably less than 0.3 g.

Soiling or spotting is prevented in particular by the fluid distribution ply being bonded to a barrier layer and/or to a backsheet ply which has a DIN 53886 hydrohead value of about 15 cm or more, although the value may also be less. The fluid distribution ply preferably comprises a nonwoven material which comprises spunbond nonwoven, spun-laced nonwoven, meltblown nonwoven, carded nonwoven, highloft nonwoven, for example highloft nonwoven from carded nonwoven, foam, preferably open-cell foam, for example foamed plastic, cellulosic fiber, mixtures of these materials in the form of intermix blends or different plies, singly or composited together.

In a further refinement, the fluid distribution ply comprises one or more plies or materials or blends. When fiber materials are used, it is preferable for the average diameter of the fibers of the fluid distribution ply to be larger than that of fibers of the backsheet ply and/or of the barrier layer.

Preferably, the fiber diameter has a value of 9 μm or more; more particularly, it is greater than 15 μm.

The ERT 40.3-90 basis weight of the fluid distribution ply is preferably in a range between 5 g/m² and 200 g/m² or more and preferably between 8 g/m² to 50 g/m² and especially in a range between 10 g/m² to 31 g/m². In the case of a sanitary napkin, a preferred basis weight is about 10 g/m² to 60 g/m². The fluid distribution ply preferably comprises at least one thermoplastic polymer or copolymer selected for example from the group consisting of polyolefins, polyamides, polyesters, polyethers and polyesterethers, the term polyolefins comprehending homopolymers and copolymers based on ethylene and/or propylene.

The table which follows lists materials which have been determined to be suitable for use in a disposable product and especially in a sanitary napkin. Table 1 is only illustrative in that no other materials shall thereby be excluded or else the indicated parameters should thereby be understood as limits. On the contrary, the indicated weights may be varied both in the upward and in the downward direction.

TABLE 1

| | | Basis weight (g/m²), |
|---|---|---|
| Ply | Type | illustrative |
| CB1 | SMMS | 16 |
| CB2 | SM | 25 |
| CB3 | M | 16 |
| CB4 | SMS | 18 |
| CBS | SMS | 24 |
| CB6 | SMS | 11 |
| SP1 | CR | 50 |
| SP2 | S (2.5 dtex) | 20 |
| SP3 | S (4 dtex) | 20 |
| BS1 | SM | 27.5, containing a fluorocarbon |
| | | additive |
| BS2 | SM | 27.5 |
| BS3 | SM | 27.5, containing an |
| | | organometallic additive |
| BS4 | SF | 33 |
| BS5 | SMSF | 44 |
| BS6 | SM | 25 |

Abbreviations: CB core barrier layer; SP spacer layer fluid and distribution ply; BS backsheet ply; S spunbond; M meltblown; CH carded highloft web consolidated by hot air; F film

In the case of SM, SMS or SMMS materials for the barrier layer between the core and the fluid distribution ply, the target spunbond weight per ply is between 3 and 25 g/m². In the case of plural plies, approximately identical basis weights can be present. In a further refinement, nonidentical basis weights are present, for example an SMS material where a first spunbond ply has a 50% to 150% higher basis weight than a second spunbond ply. The meltblown fraction is preferably between 1 g/m² and 8 g/m².

The backsheet ply utilizes for example a spunbond weight which preferably has a basis weight between 12 g/m² and 24 g/m². Any film used is microporous in particular. The film weight is preferably 15 g/m² to about 32 g/m². In a further refinement, the basis weight of the film is higher, preferably by between 12.5% and 100%, than the basis weight of the nonwoven which is likewise present.

Advantageous disposable products such as sanitary napkins result in particular in the case of the following combinations of materials:

TABLE 2

| | |
|---|---|
| Backsheet ply or barrier layer | BS1, BS2, BS4 |
| Fluid distribution and storage ply | SP2, SP3 |
| Doubled fluid distribution and storage | SP2 + SP3 |
| ply having reduced wet through | |
| Barrier layer and fluid distribution | CB2 + SP2, CB2 + SP3 |
| and storage ply having reduced wet | |
| through | |
| Fluid distribution and storage ply and | SP3 + BS6 |
| backsheet ply having reduced wet | |
| through | |

Table 3 illustrates some parameters which may be employed for a disposable product, especially a sanitary napkin, that has a fluid distribution and storage ply.

TABLE 3

| | | | Region | |
|---|---|---|---|---|
| Property | Method | CB | SP | BS |
| Air perme- | ERT 140.1- | CB < SP | >CB; BS | BS < SP |
| ability | 81 | | | |
| Basis | ERT 40.3- | 10-100 gsm | 8-300 gsm | 11-50 gsm |
| weight | 90/DIN53854 | preferably | preferably | preferably |
| | | 11-50 gsm | 10-70 gsm | 11-40 gsm |
| Fiber | CM-101-A | <SP | >CB; BS | preferably |
| fineness | | >0.5 μm | >9 μm, | between 1 |
| [μm], | | preferably | preferably | and 38 μm, |
| singly or | | 1-35 μm | >15 μm, | especially |
| mixed | | | preferably | between 1 |
| | | | between 15 | and 10 μm |
| | | | and 38 μm | and 15 and |
| | | | | 38 μm |
| Hydrohead | DIN 53886 | CB > SP, | SP < CB; BS | BS > SP, |
| | | preferably | | preferably |
| | | ≧7 cm, | | ≧7 cm, |
| | | especially | | especially |
| | | >15 cm | | >14 cm |
| Pore size | CM-126-17 | CB < SP | SP > CB; BS | BS < SP |
| Wet through | Corovin | preferably | for example | preferably |
| | test method | <1 g, | with CB or | <1 g, |
| | | especially | BS: | especially |
| | | ≦0.5 g | preferably | ≦0.5 g |
| | | | <1.0 g; | |
| | | | preferably | |
| | | | ≦0.5 g | |
| Storage | | <SP | >CB; BS | <SP |
| volume | | | | |
| Mechanical | eg Schiefer | <SP | >CB; BS | <SP |
| pressure | compresso- | | | |
| resistance | meter | | | |

CB: core barrier layer; SP: spacer layer fluid distribution and storage ply; BS: backsheet ply

A weighting of the individual properties, for example for a feminine hygiene article, especially for a sanitary napkin, is shown below in Table 4. The weighting may be different for other applications and fields of use.

TABLE 4

| Weighting | Property |
|---|---|
| 1 | Air permeability, hydrohead, wet through |
| 2 | Pressure resistance, storage volume |
| 3 | Pore size, basis weight |

Preferably, the fibers or filaments used for the fluid distribution and storage ply have a size which is dependent on the material used. Examples relating to this are shown below in Table 5.

TABLE 5

| Material/diameter | 10 μm | 35 μm | 38 μm |
|---|---|---|---|
| PP | 0.7 dtex | 8.7 dtex | 10.2 dtex |
| PET polyester | 1.1 dtex | 13.2 dtex | 15.5 dtex |

These examples hold especially for filaments and also for staple fibers.

In a further embodiment, the backsheet ply does not utilize a film but is, on the contrary, a nonwoven fabric material. The backsheet ply is therefore filmless. The nonwoven fabric material preferably comprises a hydrohead of more than 25 cm, especially of more than 35 cm and preferably of more than 40 cm. An SMS material is used for it for example. Depending on the SMS material weight, it is possible to achieve a hydrohead of more than 60 cm. It is accordingly possible, in one embodiment, to produce a diaper and also a napkin which are each filmless. Such a product may be constructed for example of a skin-face topsheet ply, followed by an acquisition ply, then the core, followed by a barrier ply, onto which a fluid distribution ply adjoins and finally a filmless backsheet ply.

In a further refinement, the fluid distribution ply has a surface tension of not more than 54 mN/m. When using a polyolefin and/or PET as a fibrous or filamentous material, the surface tension is preferably not more than 38 mN/m. The fluid distribution ply may further comprise at least one or more additives which lower the surface tension of the fluid distribution ply. Preferably, the surface tension is lowered to below 32 mN/m and preferably to below 30 mN/m at least partly on and/or in the fluid distribution ply. Useful additives include for example siloxanes, especially polydimethylsiloxanes, metal soaps and/or fluorous compounds. The surface tension is determined by Corovin test method CM-320-A along the lines of IST 80.7-95 and ASTM D2578-94. As well as lowering the surface tension by using one or more additives, it is possible to set the surface tension via an application to the fluid distribution ply.

The fibers used for the fluid distribution and storage ply may further have been surface shaped, having for example a star-shaped or otherwise polygonal cross section. The fibers may be hollow, carded, crimped, consisting of two or more materials, for example in the form of a core ensheathed by another material, in which case the two materials have different properties, for example in the form of different elongations, surface strengths, melting temperatures and so on. Furthermore, the fibers may have been fibrillated, possess a tacky surface and/or else be degradable, especially biodegradable.

The fibers used are bonded or consolidated for example by point consolidation, pressure consolidation, by impregnation, by means of bonding fibers, by spray consolidation, by means of tackifying or other adhesive materials, by pressure and/or heat application, by means of steam, water and/or by air, by needling and also by other means. The bonding or consolidation can be in accordance with a pattern and/or else be irregular.

Further advantageous embodiments and refinements are illustrated in the drawing which follows, where

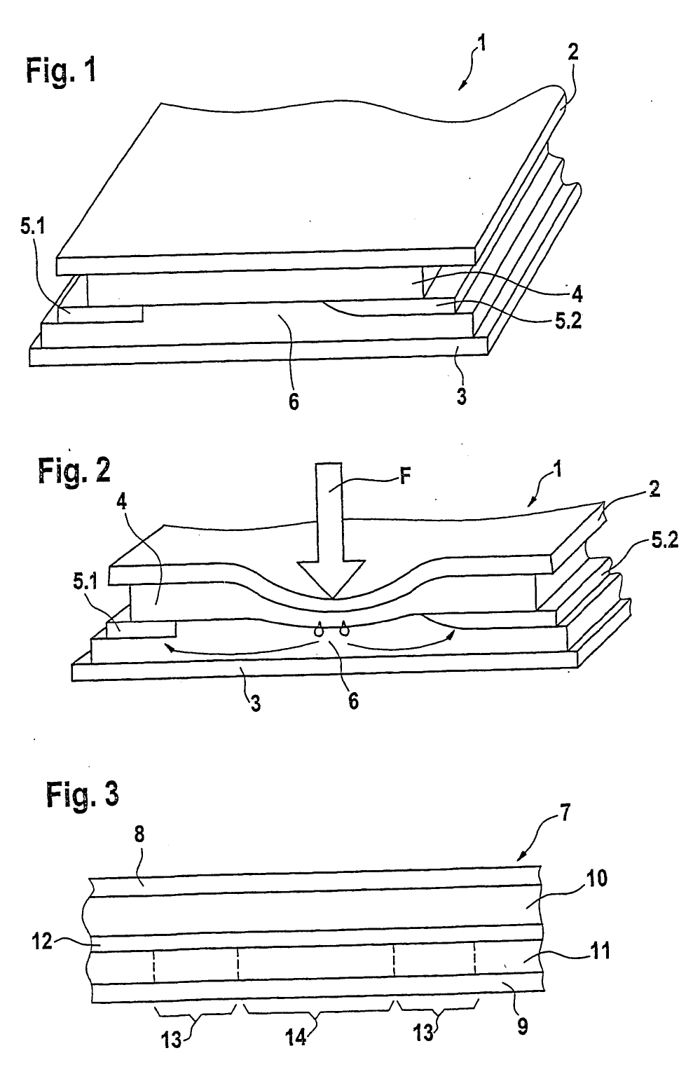

FIG. 1 shows a schematic view of a portion of a disposable article having a fluid distribution and storage ply,

FIG. 2 shows the portion of FIG. 1 under the action of a pressure,

FIG. 3 shows a further portion of a second disposable article,

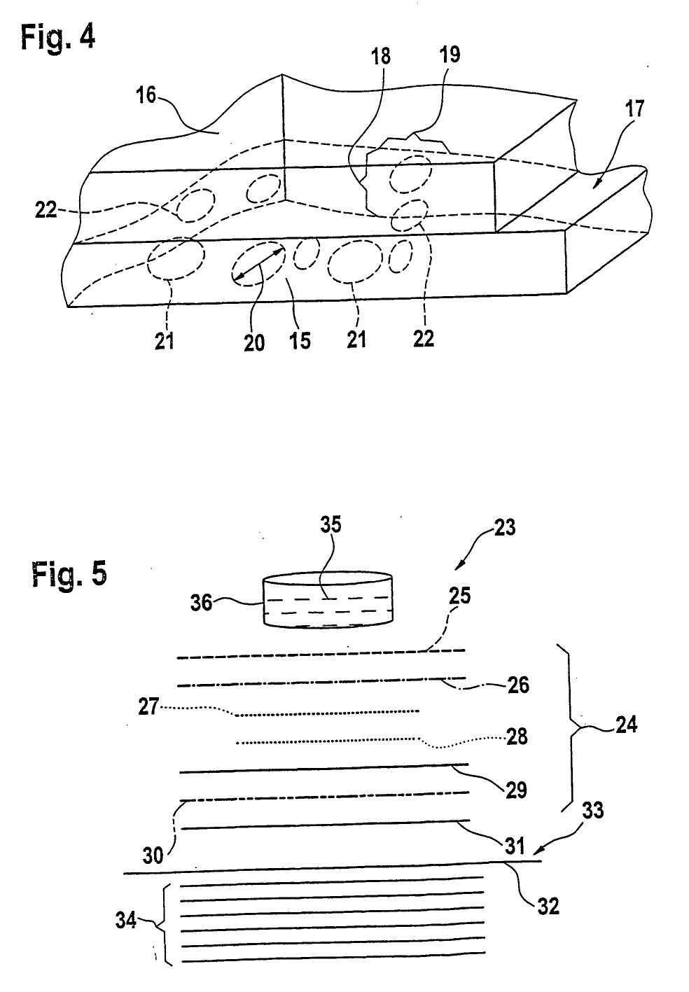

FIG. 4 shows a fluid distribution and storage ply in schematic view,

FIG. 5 shows a schematic set-up of a wet-through test,

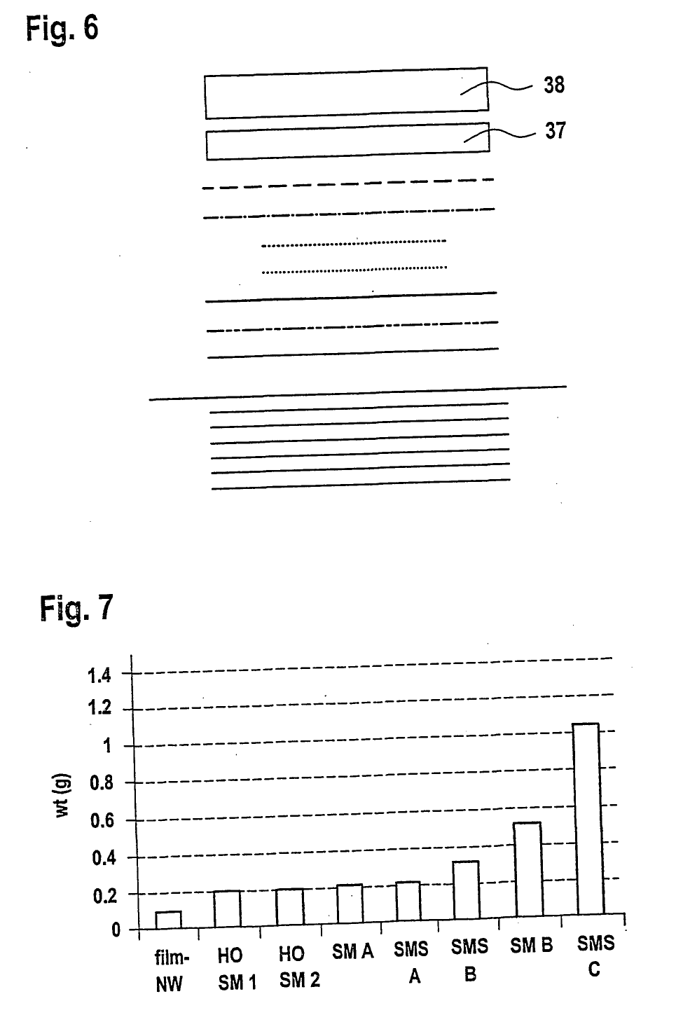

FIG. 6 shows a scematic view of the continued performance of the test according to FIG. 5.

FIG. 1 shows a portion of a disposable product 1 having a first topsheet ply 2, a first backsheet ply 3, a first core, a first barrier ply 5.1, a second barrier ply 5.2 and a first fluid distribution and storage ply 6. As depicted in FIG. 1, the first barrier ply 5.1 and also the second barrier ply 5.2 are disposed separately from the first backsheet ply 3. They are disposed as strips which protrude sideways beyond the core 4. The geometry of the first barrier ply 5.1 is such that a substantially flat ply which is the same in all directions is employed. The second barrier ply 5.2, in contrast, shows a different design. The second barrier ply 5.2 has on at least one side an arcuate surface, for example due to less material or densification of the material which is there. This is used to set a hydrostatic resistance for example. Alongside laterally disposed barrier plies 5 there may also be disposed at least one or more barrier plies further inwardly, overlappingly for example. As depicted, the first fluid distribution ply 6 likewise extends beyond the core 4 and also beyond the barrier plies 5 at two sides at least. In a further refinement, which is not depicted, the fluid distribution ply extends beyond the core 4 on all sides. It is likewise possible for the first barrier ply 5.1 and/or the second barrier ply 5.2 to be integrated in the first backsheet ply 3. In a further refinement, which is not depicted, an additional backsheet ply is present in addition to the first backsheet ply 3, between which the barrier ply 5 is disposed.

FIG. 2 shows the schematic view of the portion of the disposable product 1 of FIG. 1. On application of a pressuring force F, indicated by an arrow, on the topsheet ply 2 and/or the backsheet ply 3, the disposable product 1 deforms. Owing to the construction of the fluid distribution ply 6, a portion of the fluid present in the core 4 will flow into the fluid distribution ply 6, and is distributed sideways therein, on exceedence of a pressure limit. This process is indicated by the arrows for two drops of fluid. So, when pressure is exerted, the fluid cannot pass through the barrier ply 5 and/or the backsheet ply 3 and thereby escape. The fluid distribution ply 6 is further capable of decoupling the core 4 from the backsheet ply 3. In this refinement, the fluid distribution ply 6 decouples the core 4 from the backsheet ply 3. A locally acting peak pressure, having an impactlike action for example, will urge the fluid exiting from the core 4 quasi in the direction of the backsheet ply 3 only. The fluid will enter there and possibly even exit again on the opposite side without being able to escape sideways. This creates a continuous bridge of wetness extending from the core 4 to the backsheet ply 3. As pressure is removed, the fluid distribution ply 6 decouples this possibly resulting bridge of wetness from the core 4 and the backsheet ply 3 by interrupting the continuous band of liquid. This successfully prevents further fluid flowing, for example due to capillary action or a developing underpressure, through the backsheet ply 3. The fluid is acquired and stored in the fluid distribution ply 6.

FIG. 3 shows a further portion of a second disposable product 7. This has a second topsheet ply 8, a second backsheet ply 9, a second core 10, a second fluid distribution and storage ply 11 and an optional barrier layer 12 disposed intermediate the second fluid distribution ply 11 and the second core 10. The second fluid distribution ply 11 comprises a first territory 13 of preferred pressure application where the hydrostatic resistance is higher than in a second, neighboring territory 14 of the second fluid distribution ply 11. The second territory 14 is bounded on each of two sides by a first territory 13, as may be the case for example for a disposable product which has been conformed to a human pelvis shape. The first territory 13, the second territory 14 and also the second fluid distribution ply 11 itself in the disposable product may be disposed therein in stripe form or else uninterruptedly, for example as a cuff for extremities, as reinforcement, as cushioning or the like. The second fluid distribution and storage ply 11 preferably comprises territories having different hydrostatic resistances. This is also possible in the case of the second core 10. For example, a territory of high hydrostatic resistance in the second core 10 may adjoin a territory of lower hydrostatic resistance in the second fluid distribution ply 11, and vice versa.

FIG. 4 shows a third fluid distribution ply 15. This protrudes sideways beyond a third core 16. A protruding end 17 of the third fluid distribution ply 15 may be slung around the third core 16, so that it is impossible for fluid to escape sideways from the third core 16 without being taken up by the third fluid distribution ply 15. The third fluid distribution ply 15 has a thickness section 18 which has a hydrostatic resistance which is larger than a longitudinal section 19 (directly adjacent to the thickness section 18) of the third fluid distribution ply 15. The resistance is set for example via pores having a differing pore diameter 20. The pore diameter 20 computes from the square root of the cross-sectional area of the pore, divided by π. This also permits a comparison of differently shaped pores. The third fluid distribution ply 15 therefore has at least one first pore size 21 and a second pore size 22, which differ from each other. This makes it possible, for example, to control a capillary effect within the third fluid distribution ply 15. The arrangement of the pores is another way of controlling the hydrostatic resistance within the fluid distribution ply 15. The pores may have their largest cross section in horizontal or vertical direction or at an appropriate inclination. Variation of the flow resistance is possible not only via the number of pores but also via the size of the pores and the associated interspaces. Other possibilities are offered by a suitable choice of filament diameters, differing materials, addition of substances and so on.

FIG. 5 shows a schematic set-up 23 of a wet-through test. In the depicted test, a multi-ply structure 24 is constructed to have a third topsheet ply 25, an adjoining fourth topsheet ply 26, an adsorption-distribution layer (ADL) ply 27, a fourth core 28, a second barrier layer 29, a fourth fluid distribution and storage ply 30 and a third backsheet ply 31. This multi-ply structure 24 is placed on a cotton cloth 32, a smooth side 33 of the cotton cloth 32 being disposed in the direction of the multi-ply structure 24. If an SM material is present in the multi-ply structure 24, the meltblown side will face in the same direction as the smooth side 33. If a laminate with a film is used, the film side will face in the direction of the smooth side 33 or the meltblown side. Underneath the cotton cloth 32 there are six plies of filter paper 34. The third topsheet ply 25, the fourth topsheet ply 26, the cotton cloth 32 and also the filter paper 34 are square in shape and each measure 10×10 cm. The second barrier layer 29, the fourth fluid distribution and storage ply 30 and also the backsheet ply 31 likewise have a square shape measuring 12.5×12.5 cm. The third core 28 and also the ADL ply 27 are rectangular in shape and measure 10×5 cm. The filter paper 34 is a filter paper from Hollingsworth and Vose, Analytical and Industrial Filterpaper ERT FF3 “Strike through/Wetback”. The cotton cloth 32 has a basis weight of 12.5 g per square meter.

The test depicted in FIG. 5 is carried out as follows: First, the filter paper 34 and also the cotton cloth 32 are weighed out. Then, a test fluid 35, especially synthetic blood, is poured into a ring 36. The ring 36 has an internal diameter of 3 cm. 10 ml of test fluid 35 are poured into it. After a wait of three minutes, the ring 36 is removed from the surface. The test fluid, synthetically produced bloodlike fluid, is prepared according to Corovin GmbH's test method CM-117-B.

The rest of the test procedure is evident from FIG. 6. After the ring 36 has been removed from the third topsheet ply 25, a plate 37 is put in place. The plate 37 is for example translucent, preferably a plexiglass plate. A 3.5 kg weight 38 is then placed on the plate 37. The weight 38 compresses the multi-ply structure 24. After 10 minutes, the weight 38 is taken off the multi-ply structure 24. The wet-through fluid which has exited from the multi-ply structure 24 is calculated from the weight of filter paper 34 and the cotton cloth 32 before the fluid is applied and after the test has been carried out. Also, the cotton cloth is examined for any spotting. For example, spot length and spot width can be measured on the smooth side 33 in order that an averaged area may be determined therefrom. When this averaged area value exceeds a predeterminable limit value, then the tested multi-ply structure 24 is not suitable for a disposable product having the stipulated requirements with regard to nonsoiling and nonperviousness of fluid. In a further embodiment, spot length is used to decide the suitability of the structure for a specific application.

FIGS. 7 to 12 which follow reveal data on wet-through and on spotting of various materials and different constructions for the test objects. The materials mentioned in the individual figures are apparent below from Table 6.

TABLE 6

| | Function | | |
|---|---|---|---|
| Material | as | Construction | Property |
| Film- | BS (or CB) | Laminate of microporous film | High barrier |
| non- | | and nonwoven | effect |
| woven | | | |
| HO | BS (or CB) | Nonwoven: offline spunbond - | High barrier |
| SM1 | | meltblown, 28 g/m², inc. | effect |
| | | additive 1 | |
| | BS (or CB) | Nonwoven: offline spunbond - | High barrier |
| | | meltblown, 28 g/m², inc. | effect |
| | | additive 2 | |
| | BS (or CB) | Nonwoven: offline spunbond - | High barrier |
| | | meltblown, 28 g/m² | effect |
| | BS (or CB) | Nonwoven: inline spunbond - | Medium to low |
| | | meltblown - spunbond | barrier |
| | | (S:˜13 μm) 24 g/m² | effect |
| | BS (or CB) | Nonwoven: inline spunbond - | Medium to low |
| | | meltblown - spunbond | barrier |
| | | (S: ˜17 μm) 24 g/m² | effect |
| | BS (or CB) | Nonwoven: inline spunbond - | Medium to low |
| | | meltblown - spunbond | barrier |
| | | (S: ˜17 μm) 16 g/m² | effect |
| | BS (or CB) | Nonwoven: inline spunbond - | Medium to low |
| | | meltblown - spunbond 24 g/m² | barrier |
| | | | effect |
| | SP | Spunbond, 19 μm, 20 g/m² | |
| | SP | Spunbond, 24 μm, 20 g/m² | |
| | SP | Hot air consolidated, | Empty volume |
| | | highloft, 50 g/m² | |
| | SP | Lofty, calender-consolidated | Empty volume |
| | | nonwoven, 30 g/m², surface | and water- |
| | | treated | repellable |

Additives: selected from fluorine compounds, polydimethyl-siloxanes, metal soaps or others

The test results suggest that the use of a fluid distribution and storage ply makes it possible, for example, to use a weaker barrier layer on the core. The fluid distribution ply distinctly reduces the emittance of fluid and any spotting. There is a further reduction when an air-pervious backsheet ply is added adjoiningly to the fluid distribution ply. The wet-through test method detects the transmission of moisture as water vapor (WVRT) and makes it possible to test different combinations of materials. The test makes it possible to classify the quality of a fluid distribution ply when combined with further plies such as a barrier ply and/or a backsheet ply.

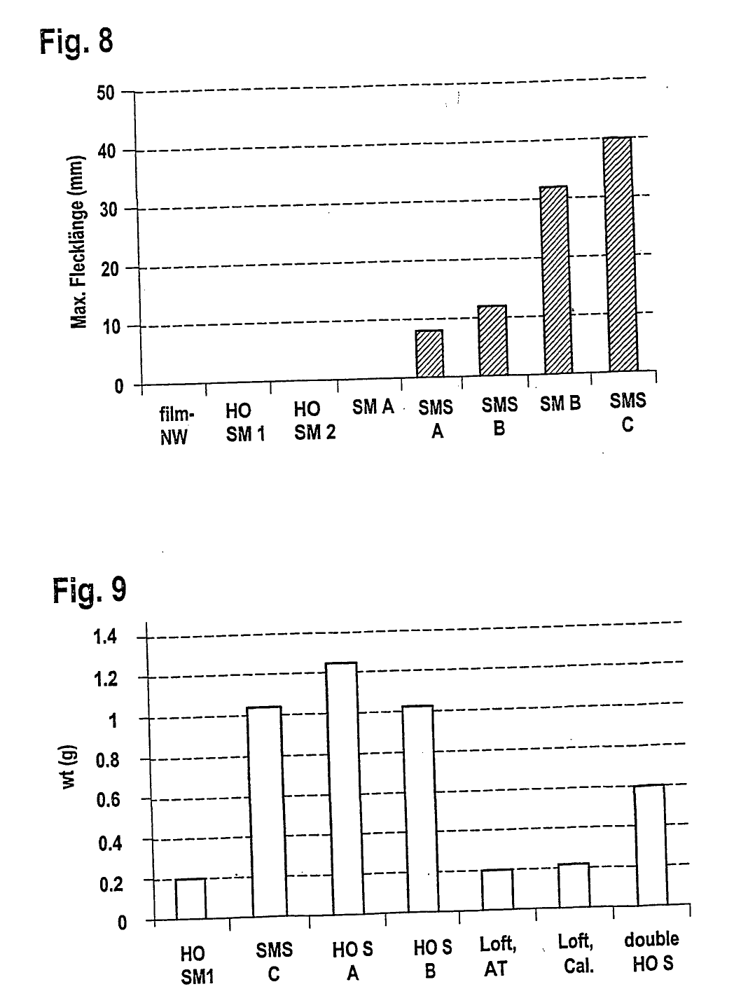

FIG. 7 and FIG. 8 show the behavior of barrier materials which were each tested singly to determine their different qualities or, to be more precise, transmission rates. To this end, the barrier materials were disposed underneath a core before the above-described wet-through test was carried out. The materials exhibit a high barrier effect from the film-nonwoven laminate to the SMA laminate. Wet-through is accordingly low. No spotting was detectable. The SMS A laminate to the SMS C laminate, by contrast, were observed to have only a minimal to moderate barrier effect. First, higher wet-through values occurred. Secondly, distinct spots were observed.

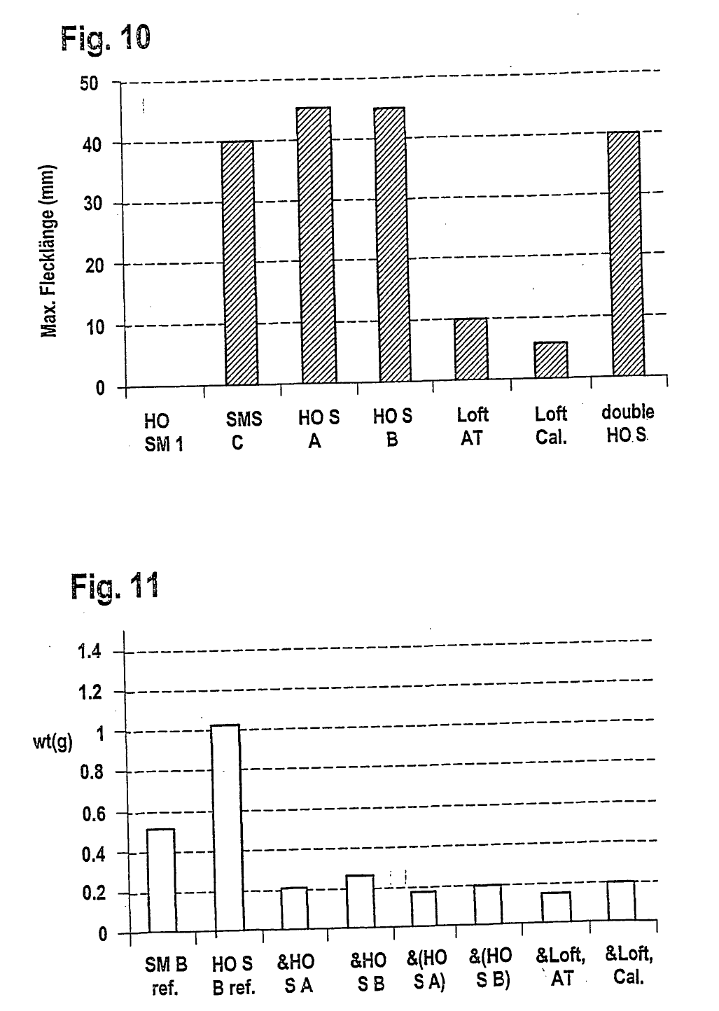

FIG. 9 and FIG. 10 show the values obtained with regard to wet-through and with regard to spotting for an arrangement where a fluid distribution and storage ply is disposed directly underneath a core. The reference sample used was HO SM 1. The data provide information as to any openness of the structure of the particular materials tested and also about the free volume contained in the materials. It is clear that there is a distinct structural difference for example between the reference sample and also SMS C, HO S A and HO S B. Wet-through is in some instances five times larger. Also tested was a doubled HO S ply, where wet-through was only three times as large however. Spotting, on the other hand, hardly differed from SMS C, HO S A and HO S B. Very low wet-through was possessed by Loft AT and Loft Cal, and spotting was distinctly less in the case of Loft Cal compared with Loft AT. However, spotting was so minimal that such materials are for example usable in a hygiene product in direct bonding to a core without barrier material in between.

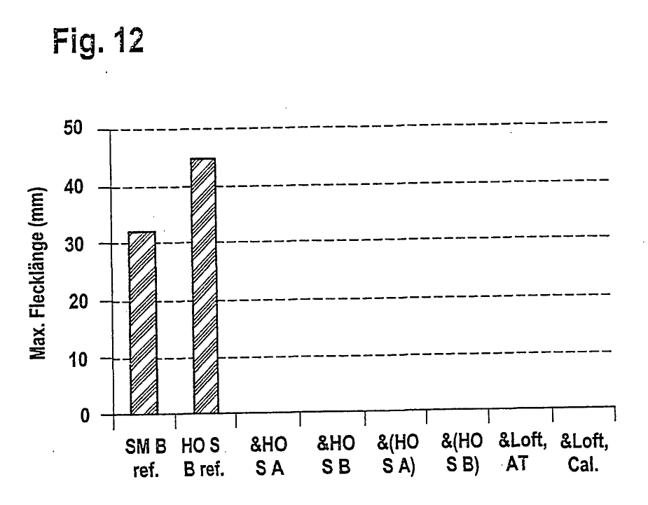

FIG. 11 and FIG. 12 show the test results for a multi-ply construction featuring a core adjoined by an SM B barrier ply. It in turn was adjoined by various fluid distribution and storage plies. Reduced emission of fluid from the core due to the barrier ply was observed. The fluid distribution ply adjacent to the barrier ply was capable of taking up, distributing and storing the fluid which had passed through. Below a certain measurement for the wet-through value, no spotting was observed. The emission of moisture which is observed was based on water vapor. In the case of the example depicted in FIGS. 11 and 12, the limit was a wet-through value of 0.2 gram. Two reference materials, which were likewise tested, are reported for the comparison. These reference materials, however, did not achieve the acquisition and distribution of fluid found for the others. Accordingly, distinct spotting was observed in the case of the reference materials.

Possible and advantageous combinations of various materials on the basis of the above results are discernible below from Table 7.

TABLE 7

| Type | Material |
|---|---|
| 1 | CB with high barrier effect + lightweight SP + |
| | BS with medium barrier effect |
| 2 | CB with medium barrier effect with medium SP + |
| | BS of medium barrier effect |
| 3 | CB of medium barrier effect + heavy/thick SP + |
| | BS of slight barrier effect |
| 4 | CB of light barrier effect + heavy/thick SP + |
| | BS of medium barrier effect |
| 5 | Heavy/thick SP + BS with high barrier effect |
| 6 | Heavy/thick SP + BS with medium barrier effect |

CB core barrier ply; SP spacer layer fluid distribution and storage ply; BS backsheet ply

The behavior of any selected construction is subject to various parameters of influence, which are appropriately adaptable for different applications. Influences on the construction include for example the number and manner of mutually overlapping plies, ply materials, the fiber size, the pore size, the thickness, the basis weight, the empty volume, the fiber orientation, the air permeability or perviousness, the surface chemistry and/or other influences.

Examples of individual parameters of influence may be as follows:

a) Mutually Overlapping Plies

The fluid distribution ply is on at least one side smaller than, equal in size to or larger than another ply.

b) Fiber Size

The fiber diameter can be chosen with different lengths, for example for staple fibers (of finite length) or filaments (of continuous length), for the barrier ply, the backsheet ply or a film.

c) Thickness

For fluid distribution ply for example the thickness is preferably more than 50 μm and especially up to 3000 μm.

d) Empty Volume

The fluid distribution ply is a structure formed from fiber to have a preferably low density but a substantial thickness. This confers on the fluid distribution ply a large free volume which can take up fluid.

e) Fiber Orientation

Fiber orientation in the nonwoven, ie the machine direction/cross direction ratio, which is measurable as a mechanical strength in the direction of and at right angles to the manufacturing direction. For example, the following MD:CD ratio from the following range is set: 0.1:1 to 30:1, preferably 1:1 to 10:1 and especially 1:1 to 5:1.

f) Air Permeability

Air pemeability or perviousness corresponds to a volume of air able to flow through the structure. Air permeability in this sense is more than just breathability (water vapor transmission rate WVRT). The air permeability of the fluid distribution ply should be more than 10 m³/m²/min for example. Further parameters of influence are included in Table 3 for example.

The fluid distribution plies in the disposable product are useful for example in hygiene products such as diapers, especially for neonates in the first four weeks, incontinence articles, napkins, sanitary products, cleaning products, wound contact materials, wound compresses, hospital articles such as drapes for operating tables, beds, bed supports and the like and also in the food sector, for example in food packaging. In a further refinement, the disposable product is also repeatedly usable. Moreover, a product may comprise a third distribution ply and a core in certain sections only, for example in the case of through-passages or enclosures which are intended to be very liquid-tight.

List of reference numerals

| | |
|---|---|
| 1 | Portion of disposable product |
| 2 | First topsheet ply |
| 3 | First backsheet ply |
| 4 | First core |
| 5 | Barrier ply |
| 6 | First fluid distribution and storage ply |
| 7 | Portion from a second disposable product |
| 8 | Second topsheet ply |
| 9 | Second backsheet ply |
| 10 | Second core |
| 11 | Second fluid distribution and storage ply |
| 12 | Barrier layer |
| 13 | First territory of preferential pressure |
| | application |
| 14 | Second territory |
| 15 | Third fluid distribution and storage ply |
| 16 | Third core |
| 17 | Protruding end |
| 18 | Thickness section |
| 19 | Longitudinal section |
| 20 | Pore diameter |
| 21 | First pore size |
| 22 | Second pore size |
| 23 | Test set-up |
| 24 | Multi-ply structure |
| 25 | Third topsheet ply |
| 26 | Fourth topsheet ply |
| 27 | ADL |
| 28 | Fourth core |
| 29 | Second barrier layer |
| 30 | Fourth fluid distribution and storage ply |
| 31 | Third backsheet ply |
| 32 | Cotton cloth |
| 33 | Smooth side |
| 34 | Filter paper |
| 35 | Test fluid |
| 36 | Ring |
| 37 | Plate |
| 38 | Weight |

1. A fluid-acquiring disposable product (1; 2), especially a hygiene product, having a topsheet ply (2; 8; 25, 25) and a fluid-acquiring and storing core (4; 10; 16; 28),
characterized in that
outwardly of the core (4; 10; 16; 28) there is disposed a fluid distribution and storage ply (6; 11; 15; 30) to acquire fluid which exits from the fluid-acquiring core under pressure (4; 10; 16; 28):

2. A disposable product as claimed in claim 1, characterized in that the fluid distribution ply (6; 11; 15; 30) exerts a lower flow resistance under pressure with regard to any distribution of a fluid in the fluid distribution ply (6; 11; 15; 30) compared with any flow resistance to fluid exiting from the fluid distribution ply (6; 11; 15; 30).

3. A disposable product (1; 2) as claimed in claim 1 or 2, characterized in that a ply adjoining the fluid distribution ply (6; 11; 15; 30) has a higher hydrostatic pressure resistance than the fluid distribution ply (6; 11; 15; 30).

4. A disposable product (1; 2) as claimed in claim 1, 2 or 3, characterized in that the fluid distribution ply (6; 11; 15; 30) has a larger average pore diameter (20) than a ply adjoining the fluid distribution ply (6; 11; 15; 30).

5. A disposable product (1; 2) as claimed in any preceding claim, characterized in that the fluid distribution ply (6; 11; 15; 30) has a nonunitary construction.

6. A disposable product (1; 2) as claimed in any preceding claim, characterized in that at least the fluid distribution ply (6; 11; 15; 30) has a wicking effect, especially a capillary effect.

7. A disposable product as claimed in any preceding claim, characterized in that the core has a wicking effect to be able to wick back fluid which is situated in the fluid distribution ply.

8. A disposable product (1; 2) as claimed in any preceding claim, characterized in that the fluid distribution ply (6; 11; 15; 30) comprises a flow inhibitor which reacts on contact with a fluid.

9. A disposable product (1; 2) as claimed in any preceding claim, characterized in that the fluid distribution ply (6; 11; 15; 30) projects beyond the core (4; 10; 16; 28) to the side at least.

10. A disposable product (1; 2) as claimed in any preceding claim, characterized in that a barrier layer (12; 29) is disposed between the fluid distribution ply (6; 11; 15; 30) and the core (4; 10; 16; 28).

11. A disposable product as claimed in any preceding claim, characterized in that the fluid distribution ply decouples the core from a backsheet ply.

12. A disposable product as claimed in any preceding claim, characterized in that it is a feminine hygiene article where the fluid distribution ply forms at least part of an outer ply of the feminine hygiene article.

13. A ply for a fluid-acquiring disposable product (1; 2) as claimed in claim 1, characterized in that the ply is a fluid distribution and storage ply (6; 11; 15; 30) having the features of any of claims 1 to 11.

14. A ply as claimed in claim 13, characterized in that the flow resistance is controlled via a bonding pattern.

15. A process for testing a flow distribution and storage ply (6; 11; 15; 30) for a fluid-acquiring disposable product (1; 2) as claimed in claim 1 or for a ply as claimed in claim 13, which comprises the steps of:
applying a fluid (35) to a multi-ply structure (24) which comprises the fluid distribution ply (6; 11; 15; 30), the fluid distribution ply (6; 11; 15; 30) adjoining a ply which is fluid acquiring,
applying a defined pressure of at least 2000 N/m² to the multi-ply structure (24) for a period of at least 5 minutes, and
determining at least one fluid passage into the fluid distribution ply (6; 11; 15; 30).
